(19) United States
(12) Patent Application Publication
(10) Pub. No.: US 2008/0020995 A1
(43) Pub. Date: Jan. 24, 2008

(54) Use of Cyclodextrin Complexes Containing Lipoic Acid

(76) Inventors: Martin Purpura, Bonn (DE); Wolfgang Schutt, Raesfeld (DE)

Correspondence Address: FULBRIGHT & JAWORSKI, LLP, 666 FIFTH AVE, NEW YORK, NY, 10103-3198, US

(21) Appl. No.: 11/720,350

(22) PCT Filed: Aug. 8, 2005
(86) PCT No.: PCT/EP05/08577

§ 371 Date: Jun. 8, 2007

(30) Foreign Application Priority Data
Dec. 17, 2004 (DE) 10 2004 060 914.4

(51) Int. Cl. A61K 31/724; A61P 17/00; A61P 25/00; A61P 3/00

(52) U.S. Cl. 514/58

(57) ABSTRACT

The invention relates to the use of cyclodextrin complexes containing lipoic acid, said complexes consisting of at least one representative of the series of unsubstituted α-cyclodextrin, β-cyclodextrin and γ-cyclodextrin, and at least one representative of the series of racemic α-lipoic acid, racemic dihydrolipoic acid and the derivatives thereof, for producing a food supplement, a functional food supplement, a clinical nutrition product and/or a cosmetic preparation in the non-medical field of application. Said complex, which contains at least 20 wt. % of the cyclodextrin component, preferably in food quality, can also contain water and reaction products or secondary reaction products from the complex production, especially α-cyclodextrin being used. The cyclodextrin/(dihydro)liponic acid complexes can be used, preferably combined with other bioactive components, aromas and/or texturing products, and also the standard formulation auxiliary agents, to produce an agent for preventing and/or treating symptoms of an inflammatory nature and especially arthritis, damage to the liver function and especially alcohol intoxication, paraesthesiae and neuropathies, and for producing agents having cytoprotective and/or antiphlogistic and/or antinociceptive (analgesic) properties and/or properties that counteract the formation of radicals. Said complexes can also preferably be used to produce an agent for stimulating glucose metabolism and/or transport in muscles and/or lipocytes, agents for reinforcing the energy metabolism of the body and the brain, agents for increasing the endurance, capacity, attentiveness, concentration of the memory, agents for the prevention and/or treatment of diabetes Typ2, and agents for caring for at least partially keratinised parts of the body, especially the skin, hair, fingernails and toenails. The invention enables products to be obtained, which can be taken by the end consumer under improved compliance conditions in the non-medical field of indication, due to the distinctive stability of the agents.

The present invention relates to novel uses of cyclodextrin complexes containing lipoic acid consisting of at least one member of the series of unsubstituted α-, β- and γ-cyclodextrin, and also at least one member of the series of racemic α-lipoic acid and racemic dihydrolipoic acid in the non-medical and medical sector.

α-Lipoic acid (thioctic acid) is a 1,2-dithiacyclopentane-3-valeric acid which exists both in racemic form and also in the form of the (R) and (S) enantiomers. α-Lipoic acid is an important component of cellular metabolism and may therefore also be found in numerous plants and animal organisms. It acts, inter alia, as one of the coenzymes in the oxidative decarboxylation of pyruvate and other α-keto acids.

For a relatively long time, α-lipoic acid has been used for prevention and therapy of various disorders, wherein, especially, liver disorders and liver damage and also diabetic and alcoholic polyneuropathies and changes of peripheral nerves which are due to metabolic disorders play a chief role.

Dihydrolipoic acid is chemically taken to mean di-6,8-dimercaptooctanoic acid, which is the reduced form of α-lipoic acid.

In addition to the pure acid forms of α-lipoic acid and dihydrolipoic acid, numerous derivatives such as esters and salts are also known.

Cyclodextrins are cyclic oligosaccharides which are made up of 6, 7 or 8 α(1-4)-linked anhydroglucose units, the best known being the α- , β- or γ-cyclodextrin variants produced by an enzymatic starch conversion and which principally differ in the diameter of their hydrophobic cavities and which are suitable in particular for the inclusion of substances of predominantly lipophilic character.

Inclusion compounds which use cyclodextrin support material are well known from the prior art.

For instance, in the German laid-open application DE 102 53 042 A1, a cosmetic preparation is described which contains a complex of cyclodextrin and vitamin F. The cyclodextrin component in this case is selected from the series of α-, β- and γ-cyclodextrin.

German laid-open application DE 102 00 657 A1 describes a 2:1 complex consisting of α- or γ-cyclodextrin and α-tocopherol. This complex can be used in cosmetic formulations and has a markedly higher stability than the corresponding physical mixtures.

The two European patent applications EP 654 484 A2 and EP 1 063 241 A1 in each case relate to drug preparations which, in addition to lipoic acid or dihydrolipoic acid, contain cyclodextrins in the form of inclusion compounds. The inclusion compounds described in these two documents are used solely for producing drugs and contain either lipoic acid or dihydrolipoic acid and a substituted cyclodextrin or else cyclodextrin or cyclodextrin derivatives in combination with the enantiomers of lipoic acid or dihydrolipoic acid. It was found that the corresponding inclusion compounds are very stable. Preferred usage forms of the medicaments described are granules, chewing tablets or effervescent tablets.

These medicaments are produced by suspending the lipoic acid component in water, then the cyclodextrin component is added and the entire reaction system is cooled; subsequently the resultant inclusion compound is isolated.

International patent application WO 2000/64440 discloses a process for the prophylaxis of damage due to injuries of the spinal chord or disorders thereof. For the purpose of prevention, a defined amount of α-lipoic acid or dihydrolipoic acid is administered to vertebrates, which preferably proceeds orally or transdermally, and for which, in particular, use is made of a cyclodextrin inclusion complex. The claimed administration is used in the medical sector in combination with what are termed decompression sicknesses (DCS).

As already stated, the cyclodextrin/lipoic acid complexes known from the prior art are distinguished by their excellent stability.

In this context, Tong Lin-Hui et al. (Journal of Inclusion Phenomena and Molecular Recognition in Chemistry; 23:119 to 126, 1995) studied inclusion complexes of α- and β-cyclodextrin with α-lipoic acid, wherein, in particular, observations on DL-α-lipoic acid played a leading role. The corresponding solid complexes were obtained according to this publication by mixing equimolar amounts of cyclodextrin and α-lipoic acid in aqueous solution, wherein, in a first step, α-lipoic acid was partly dissolved in small amounts of ethanol and β-cyclodextrin was used as saturated solution, and α-cyclodextrin was used as 2.4% strength solution. The resultant precipitated solids were separated off by filtration. Since, using stability measurements with these complexes, an elevated surface activity of lipoic acid in the complexed state was apparently observed, the conclusion was drawn that possible local irritations which are caused by lipoic acid-containing drugs can be improved by lipoic acid-containing cyclodextrin complexes.

From the prior art known to date, thus lipoic acid- and dihydrolipoic acid-containing cyclodextrin complexes are known which have improved stability. These complexes, however, have previously exclusively been provided for the medical sector or for the production of drugs. The cyclodextrin complexes contained in these drugs are distinguished by a specific ratio between the cyclodextrin component and the α-lipoic acid/dihydrolipoic acid component, wherein, in addition, preferably substituted cyclodextrins are used.

The object of the present invention is to provide novel usage sectors of cyclodextrin complexes containing lipoic acid.

This object has been achieved by using lipoic acid-containing cyclodextrin complexes consisting of at least one member of the series of unsubstituted α-, β- and γ-cyclodextrin, and also at least one member of the series of racemic α-lipoic acid, racemic dihydrolipoic acid and derivatives thereof for producing a food supplement, a functional food, a composition for clinical nutrition and/or a cosmetic preparation in the non-medical field of application.

Preferred non-medical uses are stimulating the glucose metabolism and/or transport in muscle cells and/or fat cells, enhancing the energy metabolism of the body and of the brain, increasing stamina, fitness, attention, concentration and memory but also the care of at least in part keratinous body parts, in particular the skin, hair, finger nails and toe nails.

Further preferred non-medical applications are the use of the complexes as means for clinical nutrition in the context of the prophylaxis or therapy of disorders, for example for prevention and/or treatment of symptoms of inflammatory disorder spectrum, and in particular arthritis, impairments of liver function, and in particular of alcohol intoxications, of parasthesias and neuropathies, of agents having cytoprotective and/or antiphlogistic and/or antinociceptive (analgesic) properties and/or properties which counteract the formation of free radicals, and for the prevention and/or treatment of diabetes type 2. In the context of non-medical applications, the complexes can be administered as agents not requiring a prescription, for example in the form of food supplements, functional foods and agents for clinical nutrition, if appropriate together with pharmaceutical agents requiring a prescription.

A further aspect of the invention is novel medical applications of the complexes for the prevention and/or treatment of symptoms of the inflammatory disorder spectrum, and in particular arthritis, impairments of liver function, and in particular alcohol intoxications, of parasthesias and neuropathies, of agents having cytoprotective and/or antiphlogistic and/or antinociceptive (analgesic) properties and/or properties which counteract the formation of free radicals, and for the prevention and/or treatment of diabetes type 2.

The claimed use has proved to be particularly suitable for the production of an agent which is suitable for affecting the blood glucose level and/or glucagon-like peptide 1 (GLP-1) activity and/or the binding behavior between GLP-1 and the GLP-1 receptor and/or insulin resistance and/or the in vivo conversion of glucose to glycogen and/or expression of the insulin-receptor-substrate-2 (IRS-2) polypeptide and/or insulin-stimulated glucose uptake and/or hepatic glucose formation.

In addition to the abovedescribed fields of use, the use for prevention and self-medication of spectrums of disorder in particular also comes into consideration which are associated with hyperlipidemia, hypertension, obesity and overweight, arteriosclerosis and diabetes.

In addition to the high stability of these complexes, it has proved that, in addition, the customarily unpleasant odor due to the lipoic acid component can be markedly improved or completely avoided, and that, in addition, the likewise known disturbing prickliness feeling on the uptake of relatively high amounts of lipoic acid in the throat can be completely suppressed. In addition, there is the observation that in the use according to the invention of lipoic acid-containing cyclodextrin complexes in a basic environment, as caused, for example, by corresponding components in blends in the non-medical sector, or by temperature effects as a consequence of non-standardized storage, as frequently take place with end users, are extremely stable toward polymerization processes.

In particular the improvements in the form of a reduced prickliness sensation and the suppression of the otherwise disturbing odor, even without additional encapsulation of the complex or other formulation measures, lead to markedly improved compliance behavior, which, especially for the claimed use in the non-medical sector in the form of food supplements, functional foods and also in the cosmetics sector, is of great importance and was not thus to be expected.

It has proved to be particularly advantageous when a complex is used which contains at least 20% by weight of the cyclodextrin component, fractions between 30 and 99.9% by weight being considered particularly preferred; in this context, it is advisable particularly to use the cyclodextrin component in food quality, with, for the claimed use overall, α-cyclo-dextrin being particularly advisable.

Since the use according to the invention is not restricted to a defined composition, the fractions of the two main components cyclodextrin and α-lipoic acid or dihydrolipoic acid can be varied in broad ranges; however, quantitative ratios have turned out to be advisable in which the components cyclodextrin and (dihydro)lipoic acid are present in the ratio of 5 to 99:1. In the context of the present invention, complexes are considered to be preferred which contain 20 to 95% by weight, in particular 60 to 90% by weight, and especially 70 to 85% by weight of the cyclodextrin component and 80 to 5% by weight, in particular 20 to 15% by weight, and in particular 15 to 10% by weight, of the lipoic acid component.

Since the two said main components, in their totality, are thus always present in excess, possible impurities owing to the production process used in each case are of only subsidiary importance. This is also a reason that a variant of the present invention is also taken into account in which a complex is used which additionally consists of water and feed products or side reaction products of complex production.

The proposed novel use is not linked to a special production process for the complexes used in each case. However, it has proved to be advantageous when, for the use according to the invention, use is made of a complex which is produced by the following process:

First, in process step a), an aqueous solution is initially charged which is of preferably basic character, which has proved to be advantageous owing to the generally known physicochemical properties of α-lipoic acid; then, in process step b), the respective lipoic acid component is added, subsequently in step c), the cyclodextrin component is added which is preferably in powder form, and subsequently d), the solution obtained from process steps a) to c) is stirred maximally to the clear point thereof. Subsequently, in step e), the cyclodextrin/lipoic acid complex is precipitated out using a mineral acid, for which hydrochloric acid is particularly suitable. Finally, in process step f), the resultant precipitation product is dried. To set the basic character of the aqueous solution initially charged in step a), preferably an alkali metal hydroxide solution and in particular NaOH should be used. The precipitation in step e) proceeds particularly well with stirring.

Obviously, the claimed use is not restricted to complexes which have been produced by this described process. Rather, complexes also come into consideration which have been produced in a manner known per se generally from solutions or using what is termed the paste method. In this case the complexes can also be produced from concentrated aqueous cyclodextrin solutions in which the cyclodextrin concentration is between 5 and 50% by weight.

The complexes obtained in each case can be used directly as claimed, but they can also be correspondingly isolated and prepared in advance by additional filtration steps, centrifugation or drying, by grinding, sieving, sifting and granulating or tableting.

As already indicated, the respective racemic α-lipoic acid and dihydrolipoic acid can also be used in the context of the present invention in the form of suitable derivatives. Preferred derivatives of α-lipoic acid and dihydrolipoic acid are esters and salts. Salt-forming agents are, typically, basic amino acids such as arginine or lysine, physiologically compatible alkali metal or alkaline earth metal hydroxy-bicarbonates or bicarbonates, ammonium hydroxide, amines of the formula N R¹, R², R³, wherein the radicals R¹ to R³ are identical or different, and hydrogen, a C₁₋₄-alkyl or C₁₋₄-hydroxyalkyl, such as, for example, monodiethanolamine, 1-amino-2-propanol and 3-amino-1-propanol; however, compounds which also come into consideration are alkylenediamines having an alkylene chain of 2 to 6 carbon atoms, such as ethylenediamine or hexamethylenetetramine, saturated cyclic amino compounds having 4 to 6 ring carbon atoms such as, for example, piperidine, piperazine, pyrrolidine and morpholine. Salt-forming agents which are likewise suitable are N-methylglucamine, creatine, tromethamol and N-methylmorpholine.

As likewise already described, according to the invention not only can complexes be used which exclusively contain cyclodextrin and (dihydro)lipoic acid, but, in addition, further production-related water and other feed products and side reaction products. However, a variant of the present invention is also taken into account in which the claimed use serves for the production of an agent which, in addition to the cyclodextrin/lipoic acid complex, also contains further bioactive components, aromas and/or texturizing agents. Compounds to be taken into account in this case are, in particular as physiologically active component, guanidine derivatives, such as creatine, creatinol and guanidinoacetic acid, but also phospholipids, such as phosphatidylcholine, phosphatidylserine, phosphatidylethanolamine, phosphatidylinositol and phosphatidic acid; in addition, compounds which come into consideration are unsaturated fatty acids, phytosterols and natural extracts and, here, in particular from arthemisia, and also vitamins, in particular of groups C and/or E, amino acids, and of course mixtures thereof.

Of course, in the context of the present invention, use can also be made of complexes which, in addition to the described bioactive components, also additionally contain formulation aids which increase the attractiveness and compliance for the end user, in particular in the non-medical field. Those which may be mentioned here are, in particular, formulation aids of the type of aromas and colorings, but also flavor enhancers and flavor intensifiers. If, as is likewise claimed, the use for production of a cosmetic preparation plays the chief role, further components which come into consideration are silicone oils, humectants, which protect the skin or hair from drying out, what are termed emollients, that is typical care agents, gel-forming agents and emulsifiers. However, mixture with sun protection filters, self-tanning compositions and vitamins and other suitable components is also possible, as are suitable in cosmetics formulations in the form of lotions, gels, powders, masks, creams (for example water in oil or oil in water emulsions), packages, care sticks, sprays and aerosols for topical application.

In summary, it can be stated that using the present invention, in principle the known cyclodextrin-(dihydro)lipoic acid complexes could, owing to their specific components, be supplied to a novel usage in the non-medical field of application. The complexes used for this, in addition to the known stability, are distinguished by the lack of the otherwise adverse features known for the lipoic acid component such as prickliness in the throat and disturbing odors, as a result of which the likewise otherwise customary additional formulation measures such as encapsulation or other formulation measures, can be omitted. In an extremely economical manner, therefore, the corresponding complexes can be supplied to the sector of self-medication, which as is known is characterized by a particularly critical consumer behavior. In contrast to the medical field of application in which self-selection and decision making by the end user is substantially suppressed, the claimed food supplements, functional foods, agents for clinical nutrition and cosmetic preparations can be made available in such a manner that they meet the consumer's desires. In this case the administration form can be varied in wide ranges, wherein, in addition to the already described cosmetic forms for oral administration, powders, granules, dragees, drops, drinks, juices and milk products, various chocolate forms, bars, chewing gums and soft foam preparations come into consideration.

The examples hereinafter illustrate the claimed advantages of the claimed use.

### EXAMPLES

1. Production of α-lipoic Acid/Cyclodextrin Complexes

30.5 kg of NaOH were introduced with stirring into 1.0 l (1000 kg) of water. Subsequently, this basic aqueous solution was admixed with 45.5 kg of racemic α-lipoic acid and the resultant clear solution was admixed with 180.9 kg of the α-cyclodextrin component (Cavamax® W6 from Wacker Chemie GmbH). After this solution had been stirred to the clear point, as mineral acid, 22.7 kg of HCl were added with stirring and in this manner the desired complex was precipitated out. The complexing process was completed by stirring the solution for a further 40 minutes at 22° C. Subsequently the complex was separated off by filtration and dried at temperatures of 110° C.

2. Stability Studies

First, a blend formulation of the following composition was produced:

50 g of creatine monohydrate; 20 g of citric acid; 5.0 g of sodium carbonate; 15 g of sodium bicarbonate; 5.0 g of lemon aroma; 0.5 g of aspartame; 0.5 g of acesulfame K; 4.0 g of magnesium hydroxide

To this blend were added 0.2% by weight of α-lipoic acid and 1.67% by weight of a racemic α-lipoic acid/α-cyclodextrin complex, respectively. The preparations obtained in each case were packaged in the absence of air and light and stored at 20° C. and 40° C.

Table 1 below illustrates the results of this storage test:

TABLE 1
(Stability test):

| | | | | | |
|---|---|---|---|---|---|
| 1 month | 2 months | 3 months | 6 months | 9 months | 12 months |

α-Lipoic acid content (% by weight) of the blend
containing 0.2% ALA at 20° C.

| | | | | | |
|---|---|---|---|---|---|
| 0.19 | 0.17 | 0.16 | 0.14 | 0.12 | 0.1 |

α-Lipoic acid content (% by weight) of the blend
containing 0.2% ALA at 40° C.

| | | | | | |
|---|---|---|---|---|---|
| 0.16 | 0.11 | 0.09 | 0.06 | 0.03 | 0 |

α-Lipoic acid content (% by weight) of the blend
containing 1.67% ALA-CD at 20° C.

| | | | | | |
|---|---|---|---|---|---|
| 0.2 | 0.2 | 0.2 | 0.2 | 0.2 | 0.2 |

α-Lipoic acid content (% by weight) of the blend
containing 1.67% ALA-CD at 40° C.

| | | | | | |
|---|---|---|---|---|---|
| 0.2 | 0.2 | 0.2 | 0.2 | 0.2 | 0.2 |

3. Physicochemical Properties

The blend described under 2 was likewise admixed with 0.2% by weight of a racemic α-lipoic acid or 1.67% by weight of a racemic α-lipoic acid/α-cyclodextrin complex and pressed using a tableting machine to form effervescent tablets.

The effervescent tablets were stored in the absence of air and light at 20° C. and 40° C., in which case a similar stability course as in the stability example 2 was found.

It was found that the complex is not destroyed by mechanical stress.

4. Bioavailability

833 mg of a racemic α-lipoic acid/α-cyclodextrin complex produced according to example 1 (charge B) and 100 mg of a racemic α-lipoic acid (charge A) were charged into hard gelatin capsules. Six volunteer subjects (4 male; 2 female; mean age: 27.5 years) received in each case 1 capsule of charge A and of charge B on in each case three successive weeks, in the morning in the fasting state in the context of a two-fold cross-over study. Blood samples were taken at six time points via a Braunula. After the blood serum was obtained, aliquots thereof were prepared and stored in liquid nitrogen. Analysis of the serum samples gave the following result:

The respective AUCs (areas under the serum concentration versus time curve) were calculated for the individual bioavailability courses and the total AUCs compared with the mean courses, charge B being distinguished by prolonged absorption (retard effect).

The following mean AUC values were determined:

Charge A: 4.246

Charge B: 2.775

This gave a ratio of charge A to charge B of 1.5.

It was found that the galenical formulation charge B has a markedly better bioavailability (1.5 times) than the formulation charge A. Overall, the α-lipoic acid/α-cyclodextrin complex (charge B) exhibits a marked advantage compared with the uncomplexed α-lipoic acid formulation which is not only due to quantitative effects. In addition, in the case of the formulation charge B, prolonged bioabsorption can be observed.

1-9. (canceled)

10. A method comprising preparing a food supplement, functional food, composition for clinical nutrition or a cosmetic comprising a lipoic acid-containing cyclodextrin complex comprising of at least one unsubstituted α-, β- and γ-cyclodextrin and at least one lipoic acid component selected from the group consisting of racemic α-lipoic acid and racemic dihydrolipoic acid or a derivative thereof, wherein the complex is produced by
a) charging an aqueous solution, then
b) adding the lipoic acid component, subsequently
c) adding the cyclodextrin component then
d) stirring the solution obtained from process steps a) to c) maximally to a clear point thereof, subsequently
e) adding a mineral acid to precipitate the cyclodextrin/lipoic acid complex, and
f) drying the precipitated cyclodextrin/lipoic acid complex.

11. A method comprising administering or applying to a patient a sufficient amount of the food supplement, functional food, composition for clinical nutrition or cosmetic prepared by the method of claim 10 to enhance the energy metabolism of the body and of the brain, to increase stamina, fitness, attention, concentration, memory, or to care for at least in part keratinous body part selected from the group consisting of skin, hair, a finger nail and a toe nail.

12. A method comprising administering to a subject in need thereof a sufficient amount of a lipoic acid-containing cyclodextrin complexes prepared according to claim 10 to prevent or treat a symptom of an inflammatory disorder, an impairment of liver function, alcohol intoxication, a parasthesia, a neuropathy, of agents having cytoprotective or antiphlogistic or a anti-nociceptive (analgesic) property or a property which counteracts the formation of a free radical, an agent for stimulating the glucose metabolism or glucose transport in muscle cells or fat cells or an agent for the prevention or treatment of diabetes type 2.

13. The method as claimed in claim 12 wherein at least one of a blood glucose level, glucagon-like peptide 1 (GLP-1) activity, the binding behavior between GLP-1 and the GLP-1 receptor, insulin resistance, in vivo conversion of glucose to glycogen, expression of the insulin-receptor-substrate-2 (IRS-2) polypeptide, insulin-stimulated glucose uptake or hepatic glucose formation is effected.

14. The method as claimed in claim 10, wherein the complex contains at least 20% by weight, of the cyclodextrin component.

15. The method of claim 14, wherein the complex contains from 30 to 99.9% by weight of the cyclodextrin component.

16. The method of claim 12, wherein the complex contains α-cyclodextrin.

17. The method of claim 10, wherein the complex comprises from 20 to 95% by weight of the cyclodextrin component and from 80 to 5% by weight of the lipoic acid component.

18. The method of claim 10, wherein the complex comprises from 60 to 90% by weight of the cyclo-dextrin component and 20 to 15% by weight of the lipoic acid component.

19. The method of claim 10, wherein the complex comprises from 70 to 85% by weight of the cyclodextrin component and from 15 to 10% by weight of the lipoic acid component.

20. The method of claim 10, wherein the complex further comprises water and a feed product or a side reaction product formed during preparation of the complex.

21. The method as claimed in claim 10, wherein in process step a) wherein the aqueous solution is basic.

22. The method of claim 21, wherein said basic solution comprises an alkali metal hydroxide.

23. The method of claim 22, wherein said alkali metal hydroxide is potassium hydroxide.

24. The method of claim 10, wherein in the mineral acid is hydrochloric acid.

25. The method of claim 10, wherein in step e) the solution is stirred.

26. The method of claim 10, wherein food supplement, functional food, composition for clinical nutrition or cosmetic further comprises a bioactive component, a flavoring, or a texturizing agent.

27. The method of claim 10, wherein the food supplement, functional food, composition for clinical nutrition or cosmetic further comprises at least one of a physiologically active component, a guanidine derivative, a phospholipid, an unsaturated fatty acid, a phytosterol, a natural extract, a vitamin or an amino acid.

28. The method of claim 10, wherein the food supplement, functional food, composition for clinical nutrition or cosmetic comprises at least one of creatine, creatinol, guanidinoacetic acid, phosphatidylcholine, phosphatidylserine, phosphatidylethanolamine, phosphatidylinositol, phosphatidic acid, arthemisia extract, vitamin C or vitamin E.

29. The method of claim 10, wherein the cyclodextrin component is in powder form.
